(19) United States
(12) Patent Application Publication
(10) Pub. No.: US 2025/0073404 A1
(43) Pub. Date: Mar. 6, 2025

(54) THERAPEUTIC DEVICE WITH BREATHING TOOL AND FIDGET SPINNER

(71) Applicant: The Original Anchor Pty Ltd, Harrisdale (AU)

(72) Inventor: Debby Amelia Hardjadinata, Harrisdale (AU)

(73) Assignee: The Original Anchor Pty Ltd, Harrisdale (AU)

(21) Appl. No.: 18/457,330

(22) Filed: Aug. 28, 2023

(51) Int. Cl. A61M 16/00; A63H 33/00

(52) U.S. Cl. CPC A61M 16/0003; A63H 33/002

(57) ABSTRACT

A therapeutic device with a breathing tool and a fidget spinner that can rotate, where the fidget spinner is attached to the breathing tool. The breathing tool has a proximal end with an opening that allows air to pass through an air passage and the proximal end can be enclosed in the lips of a person and allows the person to pass breathing air through the opening. The fidget spinner may encircle the air passage. The breathing tool may include: a top portion having a receiving passage; and a bottom portion with an end section adjacent to a narrow section. The fidget spinner may be adapted to rotate about the narrow section, and the end section can be received within the receiving passage of the top portion. The breathing tool, narrow section, end section, top portion, and the receiving passage of the top portion may have a cylindrical shape.

### BACKGROUND OF THE INVENTION

### 1. Field of the disclosure

The present disclosure relates generally to therapeutic devices designed to aid in stress and anxiety reduction. More specifically, this pertains to an advanced apparatus that provides controlled breathing exercises integrated with additional interactive features to engage the user.

### 2. Description of the Related Art

The contemporary world, characterized by its rapid pace and multiple stressors, has witnessed a substantial rise in individuals grappling with stress and anxiety-related disorders. This prevalence has ushered in an array of devices and tools aimed at ameliorating these disorders' symptoms. Among these, breathing pendants, often referred to as “breathing necklaces” or “breathing devices,” have gained significant traction.

Breathing pendants are designed to facilitate users in performing controlled breathing exercises. By directing their breath into these devices, users can modulate their breathing patterns, fostering relaxation and mitigating feelings of anxiety. Grounded in time-honored therapeutic principles, these devices draw inspiration from various meditative practices, reinforcing their efficacy.

However, while breathing pendants can serve their primary purpose remarkably well, they might not be apt for all social or environmental contexts, for example in a more formal or communal settings, or when a mask needs to be worn. Furthermore, for individuals experiencing heightened anxiety or distress, a singular focus on breathing might be insufficient. There is an inherent need for an alternative interaction or engagement mode that can divert their focus, serving as a distraction from overwhelming feelings or thoughts.

Consequently, there emerges a need in the realm of therapeutic devices for an implement that retains the foundational principle of controlled breathing while introducing additional interactive elements. Such an device would cater to a wider array of contexts and user needs, ensuring consistent and versatile anxiety alleviation.

### SUMMARY OF THE INVENTION

A therapeutic device with a breathing tool and a fidget spinner that can rotate, where the fidget spinner is attached to the breathing tool. The breathing tool has a proximal end with an opening that allows air to pass through an air passage and the proximal end can be enclosed in the lips of a person and allows the person to pass breathing air through the opening.

The therapeutic device where the fidget spinner encircle the air passage. The breathing tool may include: a top portion having a receiving passage; and a bottom portion with an end section adjacent to a narrow section. The fidget spinner may be adapted to rotate about the narrow section, and the end section can be received within the receiving passage of the top portion.

The breathing tool, narrow section, end section, top portion, and the receiving passage of the top portion each have a cylindrical shape. The bottom portion may have the proximal end, and the top portion may have the distal end.

The therapeutic device where the union of the top portion and the bottom portion are held together with a press-fit between the end section and the receiving passage of the top portion.

The top portion includes an inner lip that stops the bottom portion from being inserted deeper into the top portion. The fidget spinner may be cylindrical in shape. The therapeutic device may include a strand that can lift the breathing tool. The strand may be a jewelry chain. The strand may be attached to the breathing tool. The strand maybe attached to the fidget spinner. The fidget spinner may use a bearing. The breathing tool and fidget spinner may be part of a bracelet. The therapeutic device where the breathing tool has a long axis, and the axis of rotation of the fidget spinner is parallel to the long axis of the breathing tool.

The therapeutic device may have the breathing tool with an airflow pathway extending from the proximal end to the distal end The device also includes a fidget spinner rotatably coupled to the breathing tool and configured to be manually rotated by a user.

The breathing tube may be cylindrical. The therapeutic device where the breathing tool has a long axis, and the axis of rotation of the fidget spinner is parallel to the long axis of the breathing tool.

The therapeutic device has a hollow conduit presenting a proximal end and a distal end, where the proximal end is structured to allow a user to channel their breath through the hollow conduit. The device also includes a fidget spinner, capable of independent manual rotation. The therapeutic device may have the fidget spinner encircle the hollow conduit.

### BRIEF DESCRIPTION OF THE DRAWINGS

FIG. 1 illustrates a therapeutic device with a breathing tool and a fidget spinner.

FIG. 2 illustrates a therapeutic device with a breathing tool, having a notch at the proximal end, and a jewelry chain attached to the breathing tool.

FIG. 3 illustrates a therapeutic device with a jewelry chain attached to the breathing tool.

FIG. 4 illustrates a therapeutic device with a jewelry chain attached to the fidget spinner using a D-Ring bail.

FIG. 5 illustrates a therapeutic device with a jewelry chain attached directly to the fidget spinner.

FIG. 6 illustrates a therapeutic device with the fidget spinner located at the distal end of the breathing tool.

FIG. 7 illustrates a therapeutic device wherein the fidget spinner is attached to a jewelry chain via a D-Ring bail.

FIG. 8 illustrates a therapeutic device with the fidget spinner mounted to the side of the breathing tool.

FIG. 9 illustrates a therapeutic device as part of a bracelet.

FIG. 10 illustrates a therapeutic device with a curved breathing tool, and the fidget spinner integrated as part of a bracelet.

FIG. 11A illustrates details of the construction of a therapeutic device.

FIG. 11B illustrates a cross-sectional view of the therapeutic device in FIG. 11.

FIG. 12 illustrates an embodiment where the fidget spinner is positioned at the end of the breathing tool.

### DETAILED DESCRIPTION

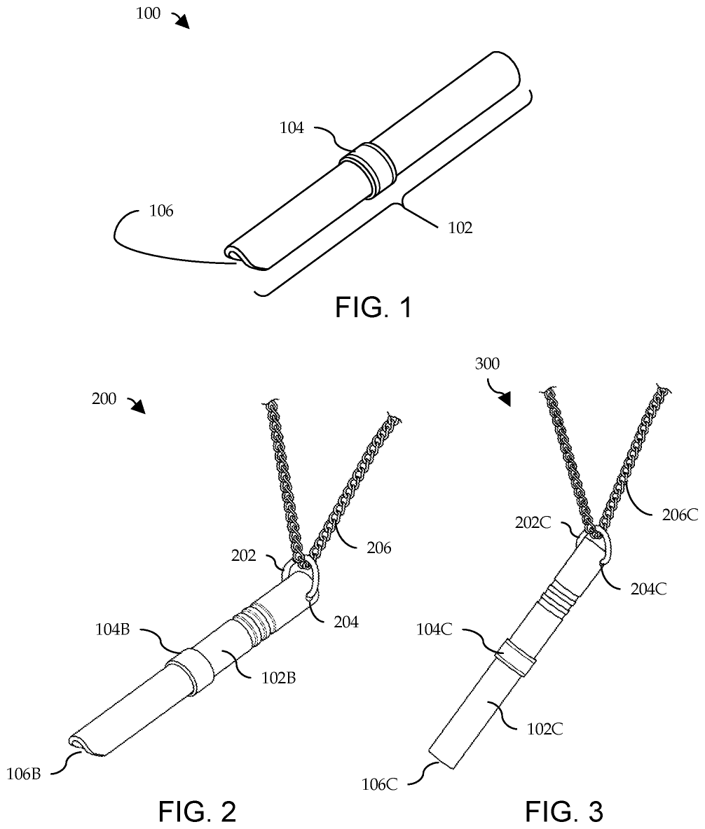

FIG. 1 illustrates a therapeutic device, designated as 100, with a breathing tool 102 and a fidget spinner 104, and where the breathing tool 102 has an opening 106 located at the proximal end of the breathing tool 102.

In use, the therapeutic device 100 is designed to enhance and regulate the breathing process. A user is recommended to inhale naturally for example drawing air in through the nose. Subsequently, the user exhales through the opening 106, taking advantage of the design for a prolonged and controlled exhalation. This method of breathing, inhaling naturally and exhaling through the device, is found to be particularly effective in regulating and stabilizing one's breath. Additionally, other breathing patterns may also be explored, such as inhaling and exhaling solely through the opening 106. By doing so, users may discover a means to further slow down their breathing, promoting relaxation and mindfulness. Different individuals might find varied patterns more beneficial based on personal comfort and objectives.

The breathing tool 102, may be various shapes and sizes. The breathing tool 102 maybe straight, curved, bent, or any other configuration. The breathing tool 102 may have a cross-sectional design can range from cylindrical and oval to square, as well as other geometric or organic shapes.

The fidget spinner 104, attached to breathing tool 102, serves as an interactive element designed to engage users. When manipulated by fingers, it can freely rotate, providing a sensory experience. The fidget spinner 104 may be located or attached anywhere to the breathing tool 102. The fidget spinner may be designed to incorporate a clearance space between itself and the breathing tool 102, ensuring smooth operation. The fidget spinner 104 may include a bearing. possibly a ball bearing, to facility the rotation of the fidget spinner 104. If a bearing is used the fidget spinner 104 may firmly connection to the breathing tool 102 and allow the outer part of the fidget spinner 104 to rotate smoothly.

Opening 106, situated at the proximal end of the breathing tool 102, allows air to pass through an air passage when a users place the proximal end between their lips. The air passage spans the length of the breathing tool 102.

FIG. 2 illustrates a therapeutic device 200 suspended from a jewelry chain 206. The device comprises a breathing tool 102B and an associated fidget spinner 104B. The breathing tool 102B has a opening 106B and holes 204, with one being visible in the current illustration. These holes 204 accommodate a D-ring bail 202, that accepts the jewelry chain 206.

The opening 106B has a notch out of the generally cylinder-shaped breathing tool 102B. The notch facilitates a breathing through the opening 106B by providing an expanded opening, ensuring easier and unobstructed inhalation and exhalation during use.

The D-ring bail 202 is connected to the holes 204, providing a reliable connection between the breathing tool 102B and a strand, exemplified by the jewelry chain 206 in this depiction. The design of the D-ring bail 202 ensures that the therapeutic device 200 remains free to swing on the jewelry chain 206. This freedom of movement prevents discomfort, such as binding or poking, when a user wears the therapeutic device 200 suspended from the chain.

The holes 204 in the breathing tool 102B serve as specific entry points designed to anchor attachments, such as the D-ring bail 202.

The jewelry chain 206 serves to hold and carry the therapeutic device, ensuring easy access for the user. It should be noted that while a jewelry chain is shown, any type of strand, such as a cord, ribbon, or leather strip, could serve this purpose. The strand could directly pass through the holes 204 and in that case the D-ring bail 202 would not be needed.

FIG. 3 illustrates a therapeutic device 300 comprising a breathing tool 102C, a fidget spinner 104C, and a jewelry chain 206C. The jewelry chain 206C is attached to the breathing tool 102C through holes 202C using a D-ring bail 204B. The proximal end of the breathing tool 102C is a straight cut of the cylindrical tube, resulting in a simple circular opening 106C.

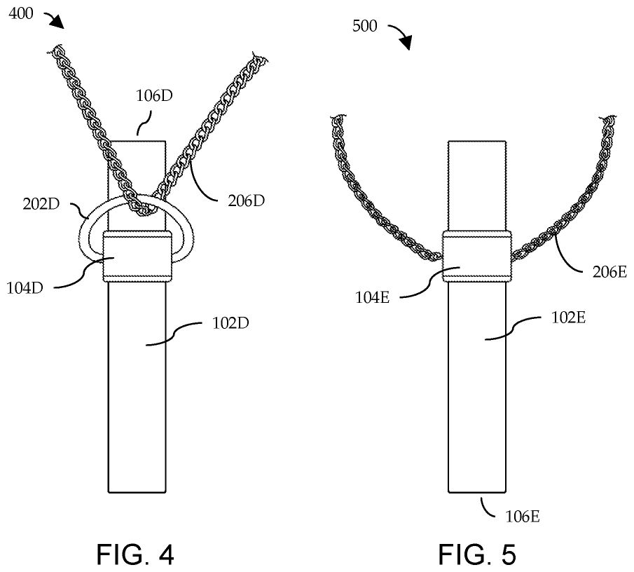

FIG. 4 illustrates a therapeutic device 400 where a jewelry chain 206D is attached to a fidget spinner 104D via a D-Ring bail 202D. The opening 106D on the proximal end is oriented upwards.

FIG. 5 illustrates a therapeutic device 500 comprising a breathing tool 102E with an opening 106E. A fidget spinner 104E is attached to the breathing tool 102E, and a jewelry chain 206E is directly connected to the fidget spinner 104E.

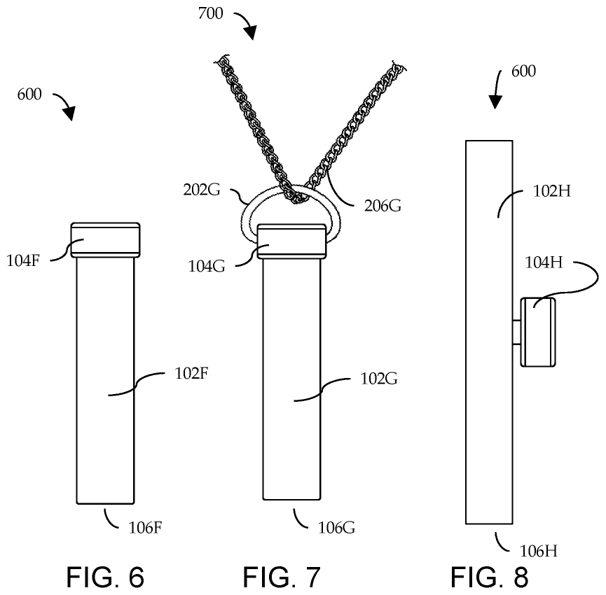

FIG. 6 illustrates a therapeutic device 600 comprising a breathing tool 102F with a fidget spinner 104F positioned at the distal end of the breathing tool 102F. The breathing tool 102F features an opening 106F situated at the proximal end of the breathing tool 102F.

FIG. 7 illustrates a therapeutic device 700 in which the fidget spinner 104G at the distal end of the breathing tool 102G. The fidget spinner 104G also attaches to a jewelry chain 206G using a D-Ring bail 204G. The breathing tool 102G has an opening 106G at its proximal end. In this version of the therapeutic device, the user may manipulate and rotate the breathing tool 102G, while the fidget spinner 104G is restricted from rotating by the jewelry chain 206G.

FIG. 8 illustrates a therapeutic device 800 where the fidget spinner 104H is mounted to the side of the breathing tool 102H. The axis of rotation for the fidget spinner 104H is orthogonal to the primary axis of the breathing tool 102H.

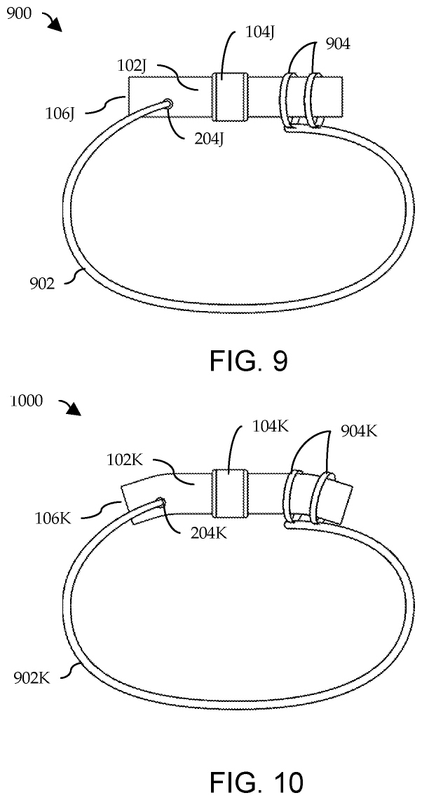

FIG. 9 illustrates therapeutic device 900, which is a bracelet with a breathing tool 102J incorporating a fidget spinner 104J. The bracelet is constructed from a material exhibiting resilience, allowing it to be pulled apart and spring back to its original form. Two prongs form a “Y” shape, with only the side view of one prong depicted. These prongs taper together into a single resilient wire which encircles a user's wrist. The two prongs connect to holes 204J (only one shown). This resilient wire continues to form loop 902 that encircles the wrist and concludes at a holster 904. The holster 904 is designed to accept the distal end of the breathing tool 102J. The proximal end of the breathing tool 102J has an opening 106J. The fidget spinner 104J remains accessible for use while the bracelet is worn. For usage of the breathing tool 102J, the user can stretch loop 902, allowing the distal end of the breathing tool 102J to be disengaged from holster 904. Once disengaged, the user can remove the bracelet from the wrist or place it on the wrist, and the breathing tool 102J becomes ready for use.

FIG. 10 illustrates a therapeutic device 1000, designed as a bracelet with a curved breathing tool 102K. The fidget spinner 104K is attached to the breathing tool 102K. Due to the curvature of the breathing tool 102K, the bracelet might offer an improved fit around the wrist. The resilient wire 902K encircles the wrist, and the holster 904K is designed to accept the distal end of the breathing tool 102K. At the proximal end of the breathing tool 102K is the opening 106K.

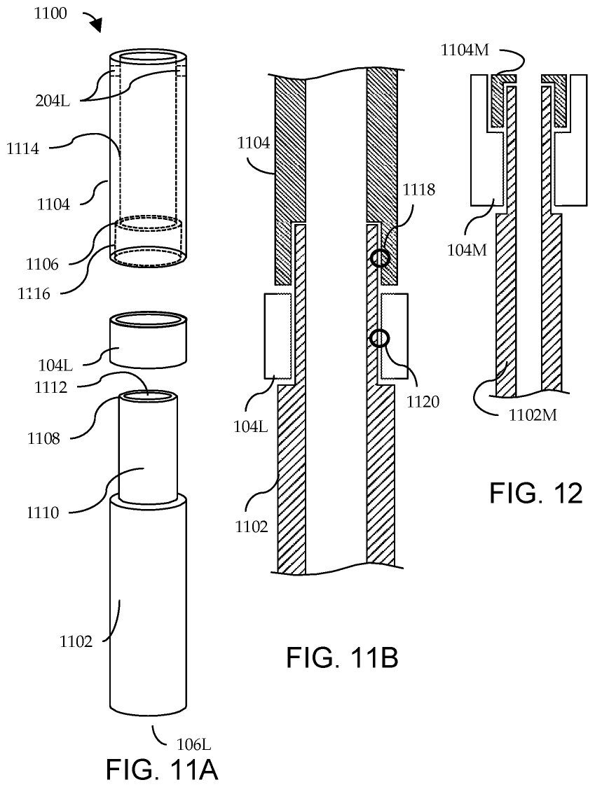

FIG. 11A displays an exploded view 1100 of a therapeutic device, with select internal components represented by dashed lines. The device comprises a breathing tool and a fidget spinner 104L. The breathing tool that includes a top portion 1104 and a bottom portion 1102.

The bottom portion 1102 is cylindrical and features an opening 106L at its base, an air passage 1112 through its core, and a narrow section 1110 positioned towards its upper end that culminates in the end section 1108.

Positioned around the narrow section 1110, the fidget spinner 104L can rotate.

The top portion 1104 has an interior. The interior has an air passage 1114, a receiving passage 1116, an inner lip 1106, and holes 204L.

The connection between the top portion 1104 and the bottom portion 1106 is achieved when the receiving passage 1116 accommodates the narrow section 1110. The inner lip 1106 is positioned to halt the upward motion of the end section 1108 within the top portion 1104.

When assembled, the top 1104 and bottom 1102 portions can be tightly held together through friction between the narrow section 1110 and the receiving passage 1116. Alternatively, other attachment methods such as adhesive, threading, or various other mechanisms can be employed. The inner lip 1106 acts as a barrier, ensuring the end section 1108 doesn't progress further into the top portion 1104 leaving enough of the narrow section exposed so the fidget spinner 104L is not pinched on the sides so it can rotate freely.

FIG. 11B depicts a cross-sectional view of the assembled therapeutic device from FIG. 11A, with consistent element numbers for corresponding components. The interface 1118 between the receiving passage 1116 and the narrow section 1110 may function as a press fit, ensuring the integrity of the breathing tool's assembly. The clearance interface 1120 provides the necessary space for the fidget spinner 104L to rotate freely around the narrow section 1110.

FIG. 12 illustrates an embodiment in which the fidget spinner is positioned at the top end of the breathing tool. The bottom portion 1102M extends most of the length of the breathing tool, while the top portion 1104M engages as previously described. The fidget spinner 104M is reconfigured to cover the shorter top portion 1104M, placing the fidget spinner 104M at the top of the breathing tool.

Variations and additions are possible to the therapeutic implement as will be apparent to those skilled in the art.

1. A therapeutic device comprising:
a breathing tool with a proximal end and a distal end, where the proximal end has an opening that allows air to pass through an air passage and the proximal end can be enclosed in the lips of a person and allows the person to pass breathing air through the opening; and
a fidget spinner that can rotate,
where the fidget spinner is attached to the breathing tool.

2. The therapeutic device of claim 1, where the fidget spinner encircle the air passage.

3. The therapeutic device of claim 2, wherein the breathing tool comprises:
a top portion having an receiving passage; and
a bottom portion with an end section adjacent to a narrow section, where the fidget spinner is adapted to rotate about the narrow section, and the end section is configured to be received within the receiving passage of the top portion.

4. The therapeutic device of claim 3, where the breathing tool, narrow section, end section, top portion, and the receiving passage of the top portion each have a cylindrical shape.

5. The therapeutic device of claim 3, where the bottom portion has the proximal end, and the top portion has the distal end.

6. The therapeutic device of claim 5, where the union of the top portion and the bottom portion are held together with a press-fit between the end section and the receiving passage of the top portion.

7. The therapeutic device of claim 1, where the fidget spinner is cylindrical in shape.

8. The therapeutic device of claim 1, further comprising a strand that can lift the breathing tool.

9. The therapeutic device of claim 8 where the strand is a jewelry chain.

10. The therapeutic device of claim 8 where the strand is attached to the breathing tool.

11. The therapeutic device of claim 8 where the strand is attached to the fidget spinner.

12. The therapeutic device of claim 1, where the fidget spinner uses a bearing.

13. The therapeutic device of claim 1, where the breathing tool and fidget spinner are part of a bracelet.

14. The therapeutic device of claim 6, where the top portion includes an inner lip that stops the bottom portion from being inserted deeper into the top portion.

15. The therapeutic device of claim 1, where the breathing tool has a long axis, and the axis of rotation of the fidget spinner is parallel to the long axis of the breathing tool.

16. A therapeutic device, comprising:
a breathing tool having an airflow pathway extending from a proximal end to a distal end, said proximal end being adapted to be enclosed by the lips of a user facilitate airflow through the airflow pathway;
a fidget spinner rotatably coupled to the breathing tool and configured to be manually rotated by a user.

17. The therapeutic device of claim 16, where the breathing tube is cylindrical.

18. The therapeutic device of claim 16, where the breathing tool has a long axis, and the axis of rotation of the fidget spinner is parallel to the long axis of the breathing tool.

19. A therapeutic device, comprising:
a hollow conduit presenting a proximal end and a distal end. wherein the proximal end is structured to allow a user to channel their breath through the hollow conduit; and
a fidget spinner, capable of independent manual rotation.

20. The therapeutic device of claim 19 where the fidget spinner encircle the hollow conduit.
